(19) United States
(12) Patent Application Publication
(10) Pub. No.: US 2022/0218117 A1
(43) Pub. Date: Jul. 14, 2022

(54) MATTRESS CARE DEVICE

(71) Applicant: LG ELECTRONICS INC., Seoul (KR)

(72) Inventors: JiHyun YOON, Seoul (KR); Hyun Shin KEE, Seoul (KR); Ki Sang JUNG, Seoul (KR); Taihong KIM, Seoul (KR)

(73) Assignee: LG ELECTRONICS INC., Seoul (KR)

(21) Appl. No.: 17/566,269

(22) Filed: Dec. 30, 2021

(30) Foreign Application Priority Data
Jan. 14, 2021 (KR) 10-2021-0005136

(51) Int. Cl. A47C 31/00; A47L 7/00; A47L 9/22; A47L 9/14; A47L 9/28; A61L 2/10

(52) U.S. Cl. CPC A47C 31/00; A47L 7/008; A47L 9/22; A61L 2202/26; A47L 9/2836; A47L 7/0061; A61L 2/10; A47L 9/1409

(57) ABSTRACT

A mattress care device includes a moving module that is moved in the longitudinal direction of a mattress and a cleaning module that is installed at the moving module and moved in the width direction of the mattress. In particular, the connection portions between two side frames of the moving module and a top frame are rotatable so that the moving module can be stably moved forward and backward.

### CROSS REFERENCE TO RELATED APPLICATION

This application claims the priority benefit of Korean Patent Application No. 10-2021-0005136, filed on Jan. 14, 2021 in the Republic of Korea, which is hereby incorporated by reference in its entirety for all purposes as if fully set forth herein.

### BACKGROUND OF THE INVENTION

### 1. Field of the Invention

The present disclosure relates to a new type mattress care device that is provided at a bed to easily care for a mattress.

### 2. Description of the Background Art

In general, a mattress is laid and used on a floor, a bed, etc., as a support for bedclothes (e.g., bedding, bedcovers, etc.).

When a mattress is used for a bed, users place the mattress on a bed frame and additionally lay or put on bedclothes on the mattress to sleep.

Such a mattress for a bed is most always installed on a bed frame, so it is required to continuously care for the mattress for cleanliness.

In the related art, mattresses are cared for through periodic washing of the mattress cover, cleaning of shaking dust off the surface of the mattresses, or using of a cleaner for bedclothes.

In particular, recently, according to Korean Utility Model No. 20-0388045, Korean Patent Application Publication No. 2009-0111632, Korean Patent No. 10-1767055, Korean Patent No. 10-1853624, Korean Patent Application Publication No, 10-2020-0067464, etc., a sterilizer having a UV-C lamp on the bottom is installed over a mattress to be movable in the front-rear direction (in the longitudinal direction of a bed) so that the mattress can be sterilized and cleaned.

However, according to the related art, when both sides of the sterilizer that is moved in the front-rear direction of the mattress are not moved at the same speed, the two sides may be twisted due to the speed difference therebetween. That is, there is a problem of poor movement such as that any one side is caught in a side of the bed frame due to twist of both sides of the sterilizer.

Further, according to the related art, there is difficulty in manufacturing because the length of the UV-C lamp should correspond to the left-right width of the mattress to sufficiently radiate UV-C to the entire mattress, so the manufacturing cost is also unavoidably high.

Further, even though the sterilizer (e.g., bed cleaner) has a suction part, a sufficient suction ability is not provided because the suction part is formed to have a length corresponding to the width of the mattress, so there is a problem that the cleaning ability is remarkably decreased.

### DOCUMENTS OF RELATED ART

(Patent Document 1) Korean Utility Model No. 20-0388045.
(Patent Document 2) Korean Patent Application Publication No. 10-2009-0111632.
(Patent Document 3) Korean Patent No. 10-1767055.
(Patent Document 4) Korean Patent No. 10-1853624.
(Patent Document 5) Korean Patent Application Publication No. 10-2020-0067464.

### SUMMARY OF THE INVENTION

The present disclosure has been made in an effort to solve various problems of the related art described above and an objective of the present disclosure is to provide a new type mattress care device that can automatically sterilize and vacuum-clean a mattress and can provide a high suction force.

Another objective of the present disclosure is to provide a new type mattress care device that can prevent restriction of movement due to inclination even if there is a speed difference between the left and right sides when the mattress care device is moved.

Another objective of the present disclosure is to provide a new type mattress care device that can achieve a sufficient sterilization effect, while making it possible to minimize the size of a UV-C lamp or the number of UV-C lamps.

According to a mattress care device of the present disclosure for achieving the objectives, both side frames of a moving module may be installed to be rotatable with respect to a top frame.

According to the mattress care device of the present disclosure, the moving module may be configured to move in the front-rear direction of a mattress and a cleaning module may be configured to move in the width direction of the mattress.

According to the mattress care device of the present disclosure, connection portions between two side frames of the moving module and the top frame are configured to be rotatable, so poor movement may be prevented even though the movement speeds of the two side frames are not the same.

According to the mattress care device of the present disclosure, since the outer surface of side walls of a bed frame has a vertical surface and an inclined surface, it is possible to prevent dirt, such as dust from being caught or collected in a gap between the bottom of the top frame and the top of the mattress.

According to the mattress care device of the present disclosure, side accommodation grooves in which side frames are accommodated are formed on the top of the rear end of the bed frame, so the side frames can be kept in close contact with the bed frame when cleaning is not performed.

According to the mattress care device of the present disclosure, a storage groove is formed on the top of the rear end of the bed frame, so the top frame of the moving module can be placed and kept therein when cleaning is not performed.

According to the mattress care device of the present disclosure, the storage groove may be recessed by the thickness of the top frame.

According to the mattress care device of the present disclosure, a cleaning accommodation groove is recessed in the storage groove, so the cleaning module can be kept without being exposed to the outside.

According to the mattress care device of the present disclosure, a communication hole that communicates with the inside of a cleaner accommodation groove is formed at (e.g., in) the bed frames, so a dirt collection envelope can be replaced even with the cleaning module stored.

According to the mattress care device of the present disclosure, the side frame may be moved by at least one moving wheel that is operated by a wheel-driving motor.

According to the mattress care device of the present disclosure, bending ends bent to face each other are formed at the upper ends of the two side frames, and both ends of the top frame are placed on and hinged to the tops of the bending ends, respectively. Accordingly, poor movement can be prevented even if the movement speeds of the two side frames are not the same.

According to the mattress care device of the present disclosure, a seat groove is recessed on the bottoms of both ends of the top frame and the bending ends may be seated in the seat grooves.

According to the mattress care device of the present disclosure, a protrusion that protrudes downward to face the outer surface of the side wall of the bed frame is formed at the lower ends of the two side frames.

According to the mattress care device of the present disclosure, a rolling member may be disposed in the inner surface of the protrusion.

According to the mattress care device of the present disclosure, a plurality of rolling members may be provided and installed in the inner surface of the protrusion sequentially in a movement direction of the moving module.

According to the mattress care device of the present disclosure, the cleaning module may be formed smaller than the left-right width of the mattress and may be movably installed on the bottom of the top frame. Accordingly, it is possible to improve a suction force even with the same output and to reduce the number of UV-C LEDs.

According to the mattress care device of the present disclosure, a rack gear may be longitudinally formed on the bottom of the top frame and a movement motor having a pinion gear may be disposed at the cleaning module. Accordingly, the cleaning module can be electronically controlled to move along the top frame.

According to the mattress care device of the present disclosure, a guide rail and a guide groove are formed on the surfaces facing each other between the top frame and the cleaning module, respectively, whereby movement of the cleaning module can be guided.

According to the mattress care device of the present disclosure, the cleaning module may include a suction unit that collects dirt by providing a suction force, whereby it is possible to remove dirt on the surface of the mattress.

According to the mattress care device of the present disclosure, the cleaning module includes a UV-C emitter that emits ultra-violet-C (UV-C), thereby being able to sterilize the surface of the mattress.

According to the mattress care device of the present disclosure, when the UV-C emitter emits light to emit UV-C, a controller controls the movement speed of the cleaning module to be lower than the movement speed when the UV-C emitter does not emit light, whereby it is possible to improve sterilization effect.

According to the mattress care device of the present disclosure, when the UV-C emitter emits light to emit UV-C, a controller controls the movement speed of the moving module to be lower than the movement speed when the UV-C emitter does not emit light, whereby it is possible to improve sterilization effect.

As described above, according to the mattress care device of the present disclosure, since the two side frames are rotatably connected to the top frame of the moving module, the two side frames can be accurately moved in the longitudinal direction of the bed frame without twisting even though a speed difference is generated between the two side frames when the moving module is moved forward and backward.

According to the mattress care device of the present disclosure, since the outer surface of the side walls of the bed frame has a vertical surface and an inclined surface, it is possible to prevent dirt from being caught in the portion where the moving module is rolled.

According to the mattress care device of the present disclosure, since the storage groove and the side accommodation grooves are formed on the top of the bed frame, the moving module can be kept not to be visually seen when the moving module is not used.

According to the mattress care device of the present disclosure, since the cleaner accommodation groove is formed in the storage groove, the cleaning module can be kept not to be exposed.

According to the mattress care device of the present disclosure, since a moving wheel that is operated by a wheel-driving motor is disposed on the bottoms of the side frames, the movement speed of the moving module can be controlled.

According to the mattress care device of the present disclosure, since the two side frames and the top frame are coupled through hinges, the top frame can be easily rotated on the hinges (i.e., by the hinges) with respect to the two side frames.

According to the mattress care device of the present disclosure, since a protrusion is formed at the two side frames, poor movement that may be caused by getting caught due to rotation of the two side frames can be prevented.

According to the mattress care device of the present disclosure, since the moving module is formed larger than the left-right width of the mattress and the cleaning module is formed smaller than the left-right width of the mattress, there is an effect that it is possible to achieve sufficient sterilization and dirt collection, even if UV lamps are minimally provided and a suction force is minimally provided.

According to the mattress care device of the present disclosure, since the cleaning module can be moved from the top frame using a rack gear and a pinion gear, the movement speed of the cleaning module can be controlled.

According to the mattress care device of the present disclosure, since the cleaning module includes the UV-C emitter, there is an effect that it is possible to sterilize the mattress by emitting UV-C.

According to the mattress care device of the present disclosure, since when cleaning is performed while UV-C is emitted, only the suction unit is operated and the movement speed is controlled to be lower than that when cleaning is performed, there is an effect that it is possible to sufficiently emit UV-C and achieve a sufficient sterilization effect.

Further scope of applicability of the invention will become apparent from the detailed description given hereinafter. However, it should be understood that the detailed description and specific examples, while indicating embodiments of the invention, are given by way of illustration only, since various changes and modifications within the scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention.

FIG. 1 is a perspective view showing an installation state of a mattress care device according to an embodiment of the present invention.

FIG. 2 is a side view showing the installation state of the mattress care device according to an embodiment of the present invention.

FIG. 3 is a front view showing the installation state of the mattress care device according to an embodiment of the present invention.

FIG. 4 is a plan view showing the installation state of the mattress care device according to an embodiment of the present invention.

FIG. 5 is a perspective view illustrating the internal structure of a moving module of the mattress care device according to an embodiment of the present invention.

FIG. 6 is an enlarged view of the part “A” in FIG. 3 for illustrating the operation structure of the moving module of the mattress care device according to an embodiment of the present invention.

FIG. 7 is a perspective bottom view showing main parts to illustrate the operation structure of the moving module of the mattress care device according to an embodiment of the present invention.

FIG. 8 is a plan view showing the state in which two side frames of the moving module of the mattress care device according to an embodiment of the present invention are formed not to rotate with respect to a top frame.

FIG. 9 is an enlarged view of the part B of FIG. 8.

FIG. 10 is a cut perspective view showing main parts to illustrate a structure in which a cleaning module is installed at the moving module of the mattress care device according to an embodiment of the present invention.

FIG. 11 is a side cross-sectional view schematically showing the internal structure of the cleaning module of the mattress care device according to an embodiment of the present invention.

FIG. 12 is a front cross-sectional view schematically showing the internal structure of the cleaning module of the mattress care device according to an embodiment of the present invention.

FIG. 13 is a block diagram schematically illustrating a control structure by a controller of the mattress care device according to an embodiment of the present invention.

FIG. 14 is a perspective view illustrating a stowing state when the mattress care device according to an embodiment of the present invention is not operated.

FIG. 15 is a side view showing the stowing state when the mattress care device according to an embodiment of the present invention is not operated.

FIG. 16 is a plan view showing the stowing state when the mattress care device according to an embodiment of the present invention is not operated.

FIG. 17 is a perspective view showing the state in which the moving module is separated from a storage part when the mattress care device according to an embodiment of the present invention is operated.

FIG. 18 is a perspective view showing the state in which the moving module is separated from a storage part when the mattress care device according to an embodiment of the present invention is operated from at a different position than FIG. 17.

FIG. 19 is a side view showing the state in which the moving module is separated from the moving module housing when the mattress care device according to an embodiment of the present invention is operated.

FIG. 20 is a perspective view showing the operation state of the moving module and the cleaning module when the mattress care device according to an embodiment of the present invention is operated.

FIG. 21 is a plan view illustrating operation routes of the moving module and the cleaning module of the mattress care device according to an embodiment of the present invention.

FIG. 22 is a plan view showing the state of the moving module when a speed difference is generated between two moving wheels when the mattress care device according to an embodiment of the present invention is operated.

FIG. 23 is an enlarged view of the part “C” of FIG. 22.

FIG. 24 is a cross-sectional view illustrating a cleaning module of a mattress care device according to an embodiment of the present invention.

FIG. 25 is a view showing the states of main parts to illustrate a movement structure between a bed frame and a moving module of a mattress care device according to an embodiment of the present invention.

### DETAILED DESCRIPTION OF THE PREFERRED EMBODIMENTS

The present invention will now be described with reference to the accompanying drawings, wherein the same reference numerals have been used to identify the same or similar elements throughout the several views.

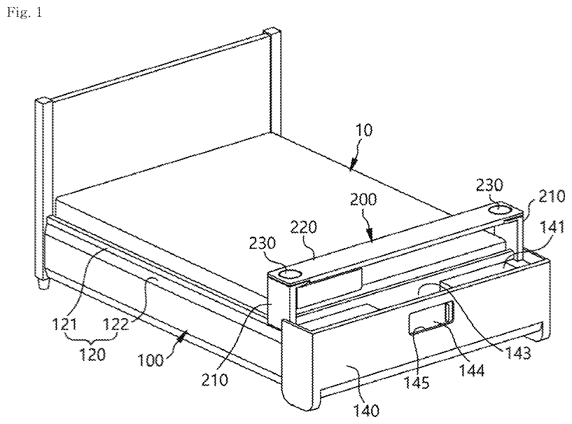

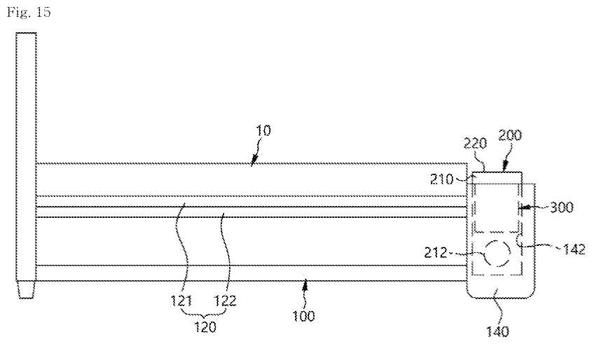

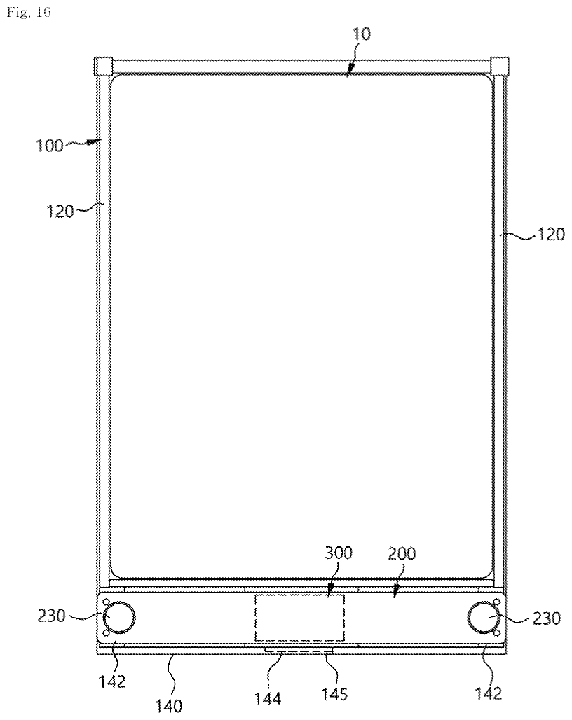

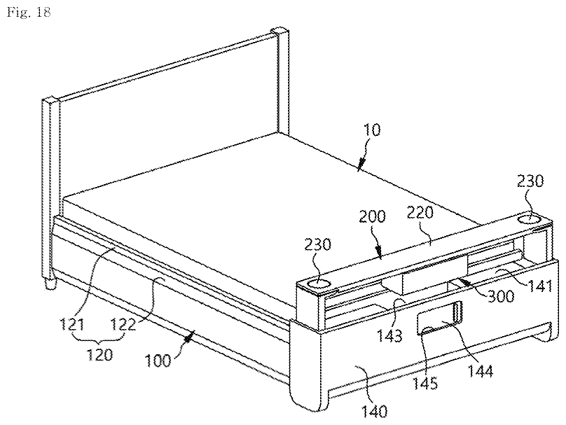

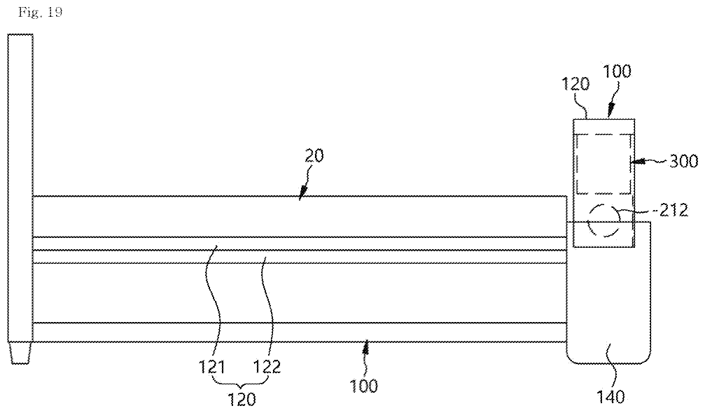

Hereafter, a mattress care device according to an embodiment of the present disclosure is described with reference to FIGS. 1 to 25.

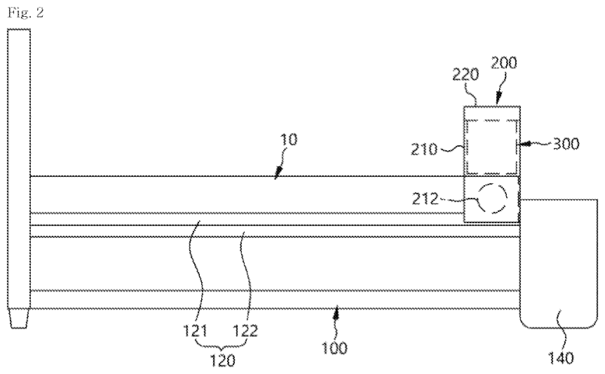

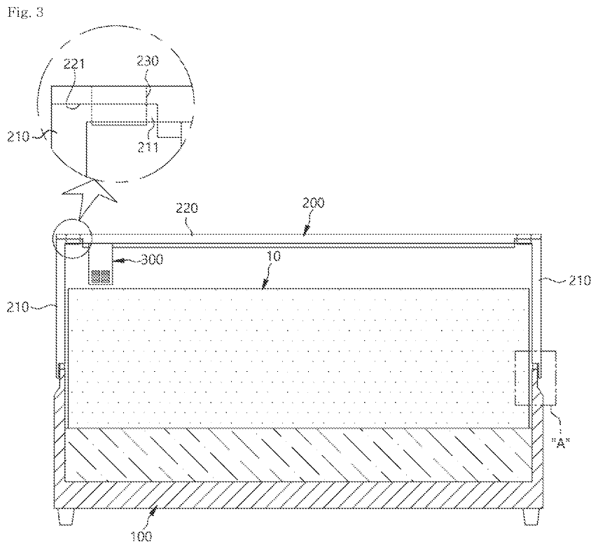

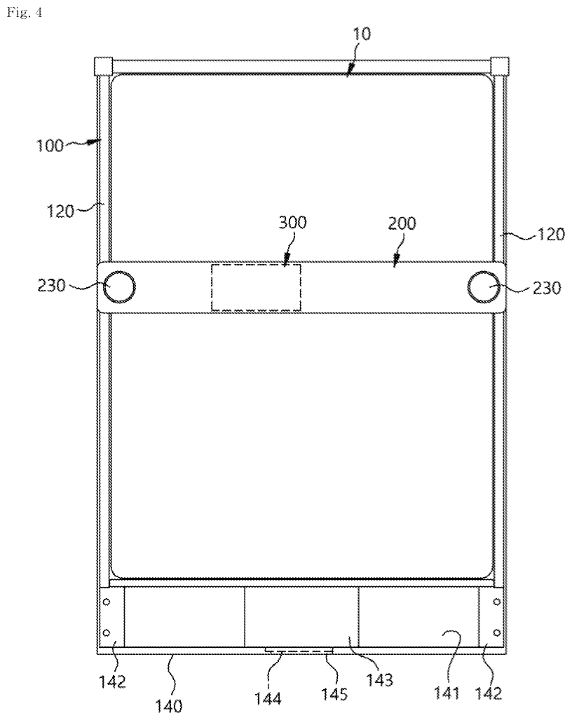

FIG. 1 is a perspective view showing an installation state of a mattress care device according to an embodiment of the present disclosure, FIG. 2 is a side view showing the installation state of the mattress care device according to an embodiment of the present disclosure, FIG. 3 is a front view showing the installation state of the mattress care device according to an embodiment of the present disclosure, and FIG. 4 is a plan view showing the installation state of the mattress care device according to an embodiment of the present disclosure.

As shown in the figures, a mattress care device according to an embodiment of the present disclosure is configured to prevent poor movement by attenuating twist of both side frames 210 of a moving module 200, which is moved in the front-rear direction of a mattress 10, due to a speed difference even if the side frames 210 are not moved at the same speed.

The mattress care device according to an embodiment of the present disclosure is described hereafter.

First, the mattress care device according to an embodiment of the present disclosure may include a bed frame 100.

The bed frame 100 is a part that forms the external appearance of a bed.

The bed frame 100 may be formed in a box shape that has two side walls 120 forming both sides and is open upward.

A vertical surface 121 and an inclined surface 122 are formed on each of the outer surfaces (e.g., surfaces exposed to the outside) of the two side walls 120. The vertical surface 121 is vertically formed from the top of the side wall 120 and the inclined surface 122 extends from the bottom of the vertical surface 121 to be inclined outward as it extends downward.

The structure of the vertical surfaces 121 and the inclined surfaces 122 are determined such that a stepped portion on which dust collects is not formed at the portions exposed to the outside of the bed frame 100. In particular, the structure of the vertical surfaces 121 and the inclined surfaces 122 can prevent the problem that dirt is caught between surfaces facing each other between the outer surfaces and the moving module 200, which is described below.

Next, the mattress care device according to an embodiment of the present disclosure includes the moving module 200.

The moving module 200 is a structure installed to be able to move in the front-rear direction of the bed frame 100 (e.g., between the head side to the foot side of the mattress, in the longitudinal direction of the bed frame 100).

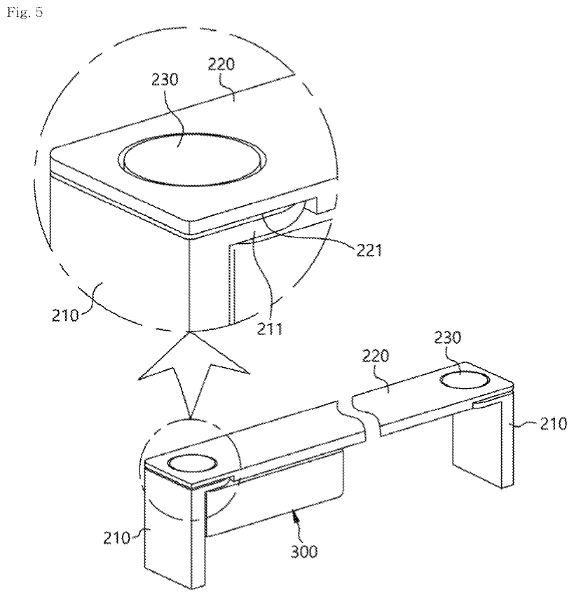

As shown in FIG. 5, the moving module 200 may include two side frames 210 and a top frame 220. That is, the top frame 220 of the moving module 200 is disposed across the top of the mattress 10 and can be moved in the longitudinal direction of the mattress 10. The side frames 210 may extend in a downward direction from the top frame 220 (e.g., from opposing first and second ends of the top frame 220), and the top frame 220 may be substantially planar.

The side frames 210 are parts forming both sides of the moving module 200.

The side frames 210 are placed on the tops (i.e., top surfaces) of the two side walls 120 of the bed frame 100 to be moved in the front-rear direction of the mattress 10 (e.g., to be moved with respect to the bed frame 100).

In particular, the upper ends of the side frames 210 are positioned higher than the mattress 10 and vertically erect (i.e., extend in a vertical direction).

A bending end 211 is formed at the upper end of the side frame 210. The bending ends 211 are bent to face each other between the two side frames 210 and are provided as portions coupled to the top frame 220. This configuration is shown in FIGS. 3 and 5. Further, each bending end 211 is connected to a respective seat groove 221 of the top plate 220, which is discussed below.

At least one rollable moving wheel 212 is installed at the bottom of each of the side frames 210. The rollable moving wheel 212 is rotated by a wheel-driving motor 213 such that the corresponding side frame 210 is moved forward or backward (i.e., along a longitudinal direction of the bed frame 100).

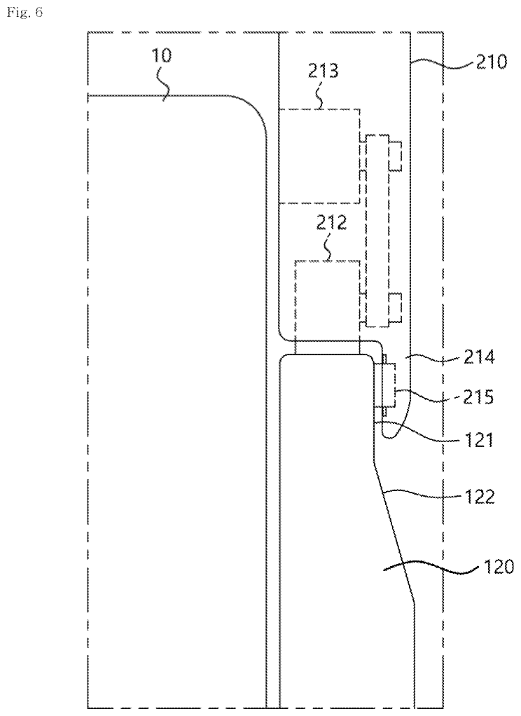

The rollable moving wheel 212 may be fitted on a shaft of the wheel-driving motor 213 or may be connected to the wheel-driving motor 213 through a chain, a belt, a gear, or the like, as known in the art, to be able to transmit power (e.g., rotational force) from the wheel-driving motor 213 to the rollable moving wheel 212, to drive the rollable moving wheel 212. This configuration is shown in FIG. 6.

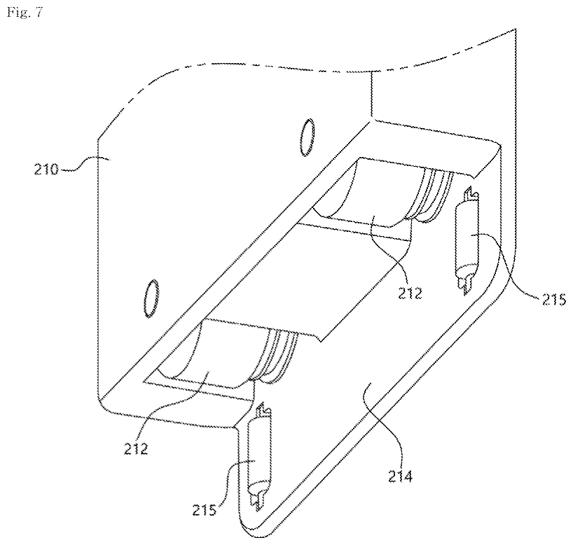

As shown in FIGS. 6 and 7, protrusions 214 that protrude downward further than the top of the two side walls 120 of the bed frame 100 are formed at the lower ends of the two side frames 210, respectively. The protrusions 214 support outer surfaces of the side walls 120 and prevent the two side frames 210 from be separated from the side walls 120.

In particular, a rolling member 215 that rolls in contact with the outer surface of the side wall 120 of the bed frame 100 may be disposed on the inner surface (i.e., the surface facing the side wall 120) of the protrusion 214.

The rolling members 215 prevent the moving module 200 from being restricted when the moving module 200 comes in contact with the side walls 120 due to twist (inclining clockwise or counterclockwise when seen from above) that is generated by a speed difference between the two side frames 210.

That is, since the two side frames 210 are operated by wheel-driving motors 213, respectively, a rolling speed difference is unavoidably generated between the rollable moving wheels 212. In this case, since the rolling members 215 are rolled in contact with the side walls 120, the moving module 200 is prevented from being restricted in the bed frame 100 (i.e., the movement of the moving module 200 with respect to the bed frame is prevented from being restricted/limited).

A plurality of rolling members 215 may be provided. In this case, the rolling members 215 may be disposed on the inner surface of the protrusion 214 sequentially in the movement direction of the moving module 200.

In detail, the rolling member 215 may be disposed at both sides of the inner surface of the protrusion 214. Accordingly, it is possible to prevent the moving module 100 from being restricted (i.e., in movement) regardless of the twist direction.

The top frame 220 is a part forming the top of the moving module 200.

Both ends (i.e., opposing first and second ends) of the top frame 220 are connected to the upper ends of the two side frames 210, respectively, and are disposed over the mattress 10 in a width direction of the mattress 10 (i.e., in a horizontal direction perpendicular to the longitudinal direction of the bed frame 100).

In particular, the length of the top frame 220 may be larger than the left-to-right width of the mattress 10.

The gap between the bottom of the top frame 220 and the top of the mattress 10 may be designed in consideration of the height of a cleaning module 300, which is described below. Of course, the two side frames 210 are configured such that their heights can be adjusted (for example, in a telescopic structure), whereby they may be used in common regardless of the thickness of the mattress 10.

Both ends of the top frame 220 are placed on the tops of the bending ends 211 formed at the upper ends of the two side frames 210. In more detail, seat grooves 221 in which the bending ends 221 are inserted may be recessed on the bottoms of both ends of the top frame 220. Each seat groove 221 may have a shape that corresponds to a shape of the respective bending end 211 to retain the bending end 211.

The connection portions between the top frame 220 and the two side frames 210 may be fixed to each other. That is, the bending ends 211 and the seat grooves 221 may be attached to one another. Alternative, the top frame 220 and the two side frames 210 may be integrally formed.

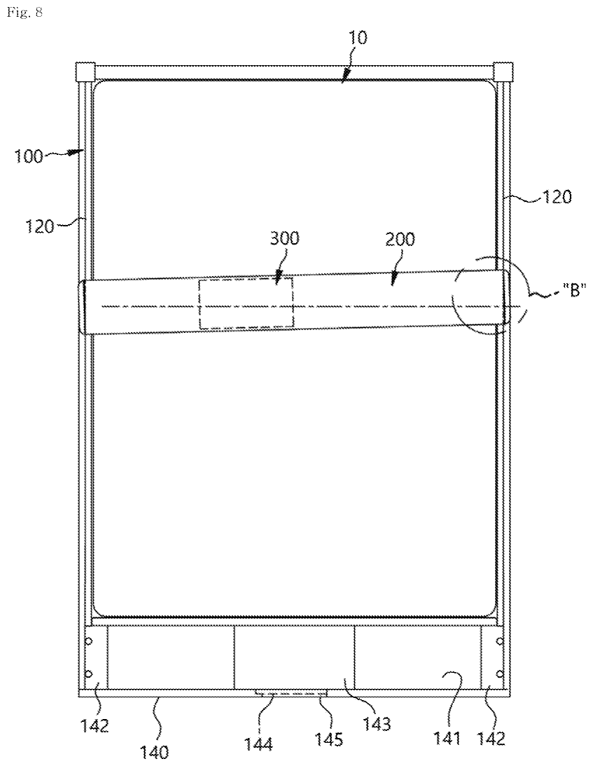

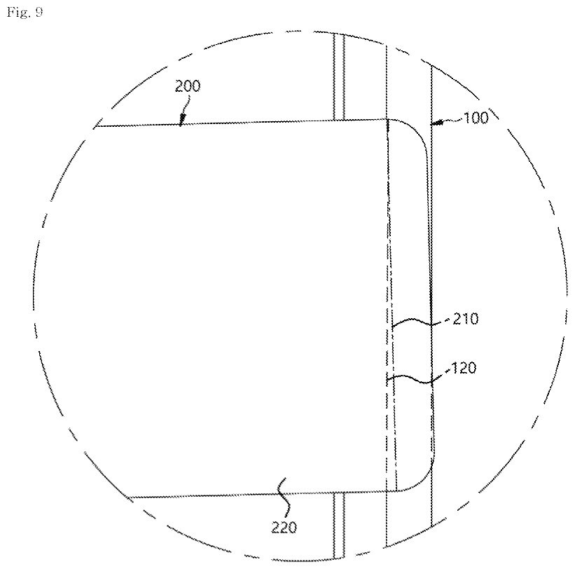

However, if the connection portions between the top frame 220 and the two side frames 210 are fixed, when there is a difference between the rolling speeds of the moving wheels at the two side frames 210, the moving module 200 twists while the side having a higher rolling speed moves faster. This configuration is shown in FIGS. 8 and 9.

In particular, when the moving module 200 twists, the side frame 210 of the faster side (i.e., the side moving first) obliquely comes in contact with the outer surface of the respective side wall 120 of the bed frame 100. Further, when the moving module 200 continues to operate to move with respect to the bed frame 100, the faster side frame 210 may be caught like a wedge (i.e., frictionally engage the respective side wall 120), to prevent further movement of the moving module 200.

Considering this problem, an embodiment of the present disclosure proposes that the two side frames 210 are rotatably coupled to the top frame 220.

That is, both ends of the top frame 220 and the bending ends 221 are coupled through hinges 230 so that the two side frames 210 can be rotated left and right (e.g., along a horizontal axis or horizontal direction) with respect to the top frame 220.

Accordingly, even if the rolling speeds of the moving wheels 212 at the two side frames 210 becomes different and any one side frame 210 gradually comes in contact with the outer surface of the side wall 120 of the bed frame 100, the side frame 210 is rotated as much as the contact force and the side frame 210 is not caught like a wedge (i.e., the side frames 210 can continue to move with respect to the bed frame 100).

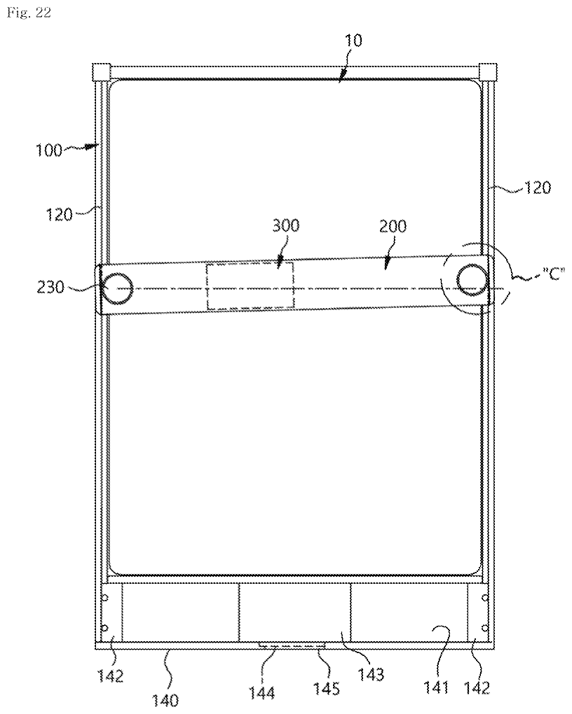

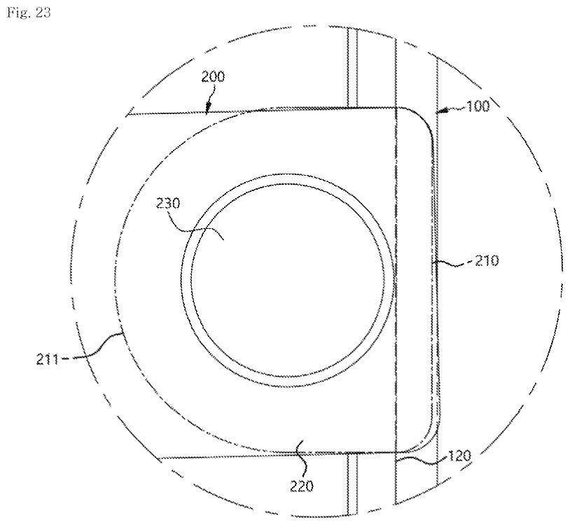

This configuration is shown in FIGS. 22 and 23. That is, a side frame 210 is rotated as much as hitting of the side frame 210 against the outer surface of a side wall 120 of the bed frame 100, whereby it is not restricted.

The hinge 230 connecting the top frame 220 and the bending end 211 may be integrally formed at at least any one of the top frame 220 and the bending end 211 and the hinge 230 may be coupled to be separable from both the top frame 220 and the bending end 211.

At least one stop position checker 240, that senses a position (e.g., of the moving module 200 in a movement direction may be disposed on at least any one of the surfaces facing each other between the moving module 200 and the bed frame 100. That is, this is for preventing power consumption by enabling the moving module 200 to move accurately within the range in which the mattress 10 is positioned without excessive moving forward or backward.

The stop position checker 240 may be a common optical sensor or proximity sensor, may be an encoder that can be installed on the moving wheel 212, or may be a switch that is installed on the surfaces facing each other between the moving module 200 and the bed frame 100.

Of course, the stop position checker 240 may be a sensor including a light emitter and a light receiver and may be disposed on the surfaces facing each other between the side frames 210 and the bed frame 100.

The stop position checker 240 may be configured to provide a sensing signal to a controller 400.

The mattress care device of the present disclosure may include a storage part 140 on the bed frame 100.

The storage part 140 is a part provided at the bed frame 100 to keep (i.e., store) the moving module 200 and the cleaning module 300 when they are not in use.

The storage part 140 may be provided at the rear end of the bed frame 100 (opposite to the headboard). That is, the moving module 200 and the cleaning module 300 can be kept at the foot side of the bed (e.g., within the foot side of the bed) to prevent users from feeling unpleasant and to be out of the way when not in use.

The storage part 140 may have a storage groove 141 in which the top frame 220 is accommodated and side accommodation grooves 142 in which the two side frames 210 are accommodated.

The storage groove 141 may be recessed from the top of the bed frame 100 and may accommodate the top frame 220. The storage groove 141 may be recessed by the depth of the top frame 220.

The side grooves 142 may be formed at both sides (i.e., opposing sides) of the storage groove 141, respectively, on the top of the bed frame 100 and may be recessed by a depth at which the side frames 210 can be fully accommodated, respectively.

A cleaner accommodation groove 143 may be recessed in the storage groove 141. The cleaner accommodation groove 143 is a part for accommodating the cleaning module 300, so the cleaning module 300 can be accommodated and kept therein.

In particular, a communicating hole 145 that communicates with the inside of the cleaner accommodation groove 143 and is opened and closed by a door 144 may be formed through a rear wall of the storage part 140 of the bed frame 100.

That is, a user can empty or replace a dirt collection tub (or dirt collection envelope) 322 in the cleaning module 300 through the communicating hole 145. Of course, an outlet hole for taking out the dirt collection tub 322 may be formed through a rear wall of the cleaning module 300.

Next, the mattress care device according to an embodiment of the present disclosure may include a cleaning module 300.

The cleaning module 300 is a device for cleaning the mattress 10.

The cleaning module 300 is installed on a bottom of the top frame 220 of the moving module 200, is formed smaller than the left-right width (i.e., in the horizontal direction) of the mattress 10, and is configured to move in the longitudinal direction of the top frame 220 (width direction of the mattress and horizontal direction/axis).

That is, the moving module 200 can be moved forward and backward with respect to the mattress 10 and the cleaning module 300 can be moved left and right with respect to the mattress 10.

Accordingly, even though the left-right width of the cleaning module 300 is smaller than the left-right width of the mattress 10, it is possible to clean all of the left and right sides of the mattress 10 and clean all of the front and rear portions of the mattress 10 in cooperation with the moving module 200.

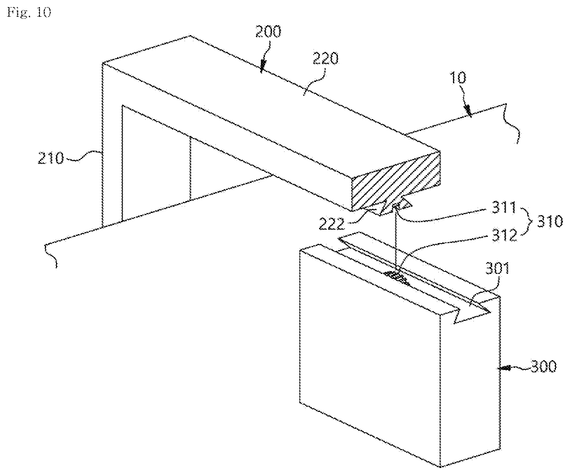

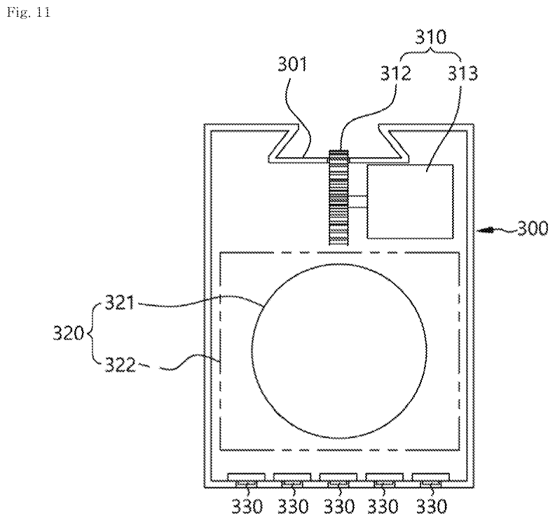

A guide rail 222 and the guide groove 301 may be formed to correspond to each other on the bottom of the top frame 220 and the top of the cleaning module 300, respectively. The guide rail 222 may have a substantially triangular shape. That is, the guide rail 222 and the guide groove 301 may be fitted to each other, whereby the cleaning module 300 can be moved to desired positions under the top frame 220. This configuration is shown in FIGS. 10 and 11.

In an embodiment of the present disclosure, it is exemplified that the guide rail 222 protrudes from the bottom of the top frame 220 and the guide groove 301 is recessed from the top of the cleaning module 300.

In particular, the guide rail 222 may be formed along the center line of the bottom of the top frame 220, and the guide rail 222 and the guide groove 301 may be formed such that both sides are gradually inclined outward as they go down. Accordingly, the cleaning module 300 can be hung on the bottom of the top frame 220 (i.e., the cleaning module 300 can be fully supported to the bottom of the top frame 220 by the connection between the guide groove 301 and the guide rail 222).

The cleaning module 300 may include a moving unit 310.

That is, a moving unit 310 that is controlled by the controller 400 is provided, whereby the cleaning module 300 can be automatically moved in the longitudinal direction of the top frame 220.

The moving unit 310 may include a movement motor 313 having a rack gear 311 and a pinion gear 312.

That is, the cleaning module 300 includes the moving unit 310, whereby the cleaning module 300 can be controlled by the controller 400 to automatically move in the longitudinal direction of the top frame 220.

The rack gear 311 may be formed on the bottom of the top frame 220 (e.g. in the center of the guide rail 222) and the pinion gear 312 may be installed/positioned on the top of the cleaning module 300 (e.g., a top exposed surface of the cleaning module 300, including within the guide groove 301) to be partially exposed. The rack gear 311 may extend along a longitudinal direction of the top frame 220. The exposed portion of the pinion gear 312 is installed to engage with the rack gear 311 and the movement motor 313 may be coupled to the pinion gear 312 through a shaft and may be disposed in the cleaning module 300.

Of course, the moving unit 310 may configured in a structure including a ball screw or an actuator in addition to the structure including the rack gear 311 and the pinion gear 312.

An intake hole 302 is formed through the bottom of the cleaning module 300 and a discharge hole 304 is formed through any one side. A filter member 303 for filtering out particulate matter in the air that is discharged may be disposed in the discharge hole 304.

A suction unit 320 may be disposed in the cleaning module 300.

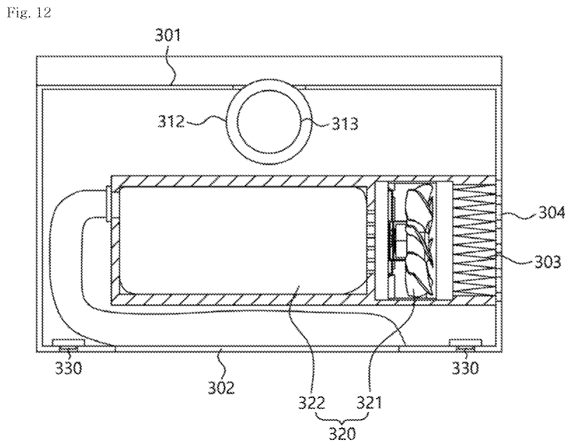

The suction unit 320 (i.e., vacuum) is provided to suction and remove dirt on the top of the bed 10 by providing a suction force, and may include a fan motor 321 disposed in the cleaning module 300 and a dirt collection tub 322 in which dirt suctioned through the intake hole 302 is collected. This configuration is shown in FIG. 12.

The dirt collection tub 322 may be a replaceable paper envelope, as shown as an embodiment. Of course, though the dirt collection tub 322 may be a tub that can put in and taken out of the cleaning module 300 (i.e., removably attached to the cleaning module 300) so that collected dirt can be removed out of the tub.

The cleaning module 300 may include at least one UV-C emitter 330 that emits ultraviolet-c (UV-C). That is, the top of the mattress can be sterilized by the UV-C emitted by the UV-C emitter 330.

The UV-C emitter 330 may be disposed at at least any one position on the bottom of the cleaning module 300 but may be disposed in multiple different positions on the cleaning module 300.

The UV-C emitter 330 may be any one of a UV-C lamp and a UV-C LED. It is exemplified in an embodiment of the present disclosure that the UV-C emitter 330 is a UV-C LED.

Of course, only any one of the suction unit 320 and the UV-C emitter 330 may be provided at the cleaning module 300 or both of them may be provided.

It is exemplified in an embodiment of the present disclosure that the cleaning module 300 has both the suction unit 320 and the UV-C emitter 330. In this case, the UV-C LED of the UV-C emitter 330 may be formed around the intake hole 302 formed on the bottom of the cleaning module 300.

Meanwhile, the cleaning module 300 may be movably installed on any one of the front, rear, and top of the top frame 220.

In this case, the rack gear 311 and pinion gear 312, and the guide rail 222 and guide groove 301 may be provided or formed on the surfaces facing each other between the top frame 220 and the cleaning module 300, respectively.

Next, the mattress care device according to an embodiment of the present disclosure may include a controller 400.

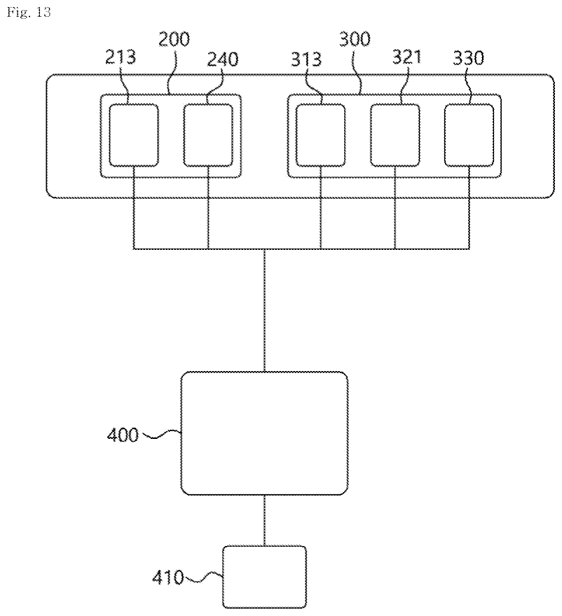

The controller 400 is, as shown in FIG. 13, configured to control the operation of the moving module 200 and the cleaning module 300. That is, the controller 400 controls the wheel-driving motors 213 to move the moving module 200 forward and backward while controlling and operates the cleaning module 300 by controlling the movement motor 313, the fan motor 321, and the UV-C emitter 330.

The controller 400 may be disposed at the moving module 200 or may be disposed at the cleaning module 300. The controller 400 may be provided at the cleaning module 300. Of course, an operation unit 410 for manually operating the controller 400 may be provided at the moving module 200.

In particular, the controller 400 may be configured to change the movement speed of the cleaning module 300 in accordance with the operation of the UV-C emitter 330.

That is, when the UV-C emitter 330 emits light to emit UV-C, the moving module 200 or the cleaning module 300 is controlled to move slower than the movement speed when the UV-C emitter 330 does not emit light.

Accordingly, UV-C can be sufficiently emitted to the mattress 10 and a sterilization effect can be achieved. Further, it is possible to reduce the cleaning time when performing only suction without sterilization.

The controller 400 can control the moving module 200, via the wheel-driving motors 213, to stop when receiving a sensing signal from the stop position checker 240 disposed on the surfaces facing each other between the moving module 200 and the bed frame 100.

Accordingly, the moving module 200 can be reciprocated between the head side and the foot side of the bed frame 100.

The process of cleaning the mattress 10 using the mattress care device of the present disclosure described above is described hereafter in more detail.

First, when the mattress 10 is not cleaned, the moving module 200 of the mattress care devices is positioned and kept at the rear portion (foot side) of the mattress 10.

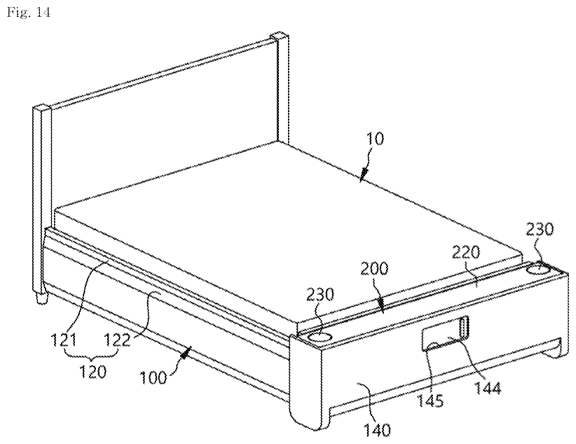

In more detail, the moving module 200 may be kept in the storage part 140 when cleaning is not performed. This configuration is shown in FIGS. 14 to 16.

In particular, the moving module 200 may be positioned at the center portion of the bed manually by a user or by operation of the controller 400 through the operation unit 410 and then used as a bed table, (e.g., as a desk).

When a request for cleaning the mattress 10 is generated in the initial non-using state, the controller 400 controls the moving module 200 and the cleaning module 300 to clean the mattress 10.

The request for cleaning the mattress 10 may be input (or operated) manually through the operation unit 410 by a user or may be automatically input at a predetermined cycle (or time).

In particular, when a request for cleaning the mattress 10 is generated, the controller 400 controls the moving module 200 to move to the rear (i.e., foot side) or the front (i.e., head side) of the bed frame 100 and controls the cleaning module 300 to move to any one side of the top frame 220. These positions of the moving module 200 and the cleaning module 300 may be positions set in advance (i.e., set positions) for automatically perform cleaning.

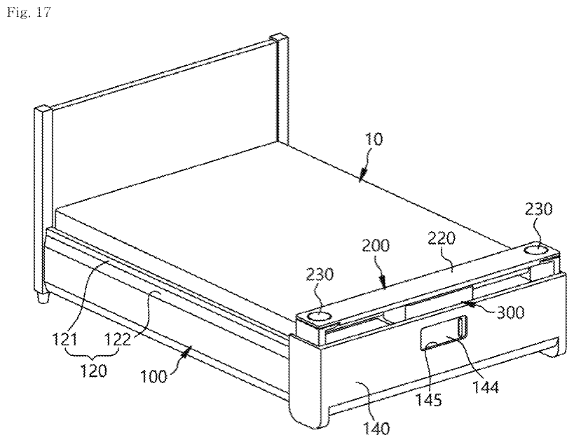

FIGS. 17 to 19 show the state in which the moving module 200 and the cleaning module 300 come out of the storage part 140 and move to the set positions.

It is possible to recognize whether the moving module 200 has returned to the set position through the stop position checker 240.

When the moving module 200 and the cleaning module 300 return to the set positions, the controller 400 controls the moving module 200 to move forward by operating the wheel-driving motors 213.

While the moving module 200 moves, the controller 400 controls the suction unit 320 of the cleaning module 300 to operate.

Accordingly, the top of the mattress 10 is first suctioned into the cleaning module 300 and collected in the dirt collection tub 322.

Further, the controller 400 operates the movement motor 313 of the cleaning module 300 when the moving module 200 moves forward, such that the cleaning module 300 moves in the longitudinal direction of the top frame 220.

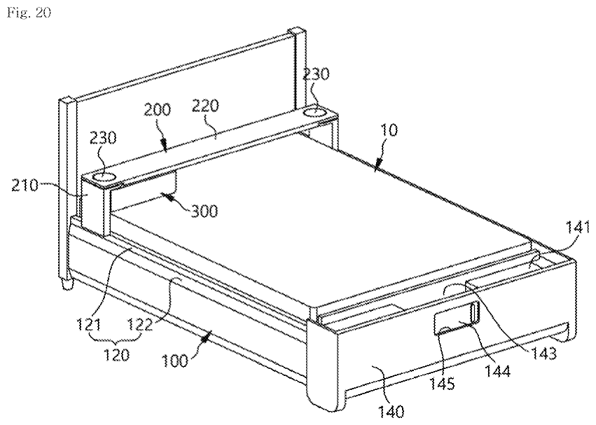

Accordingly, the cleaning module 300 suctions dirt on the top of the mattress 10 while being supported by the guide rail 222 and moves from a first side to an opposite second side (see FIG. 20) of the top frame 220.

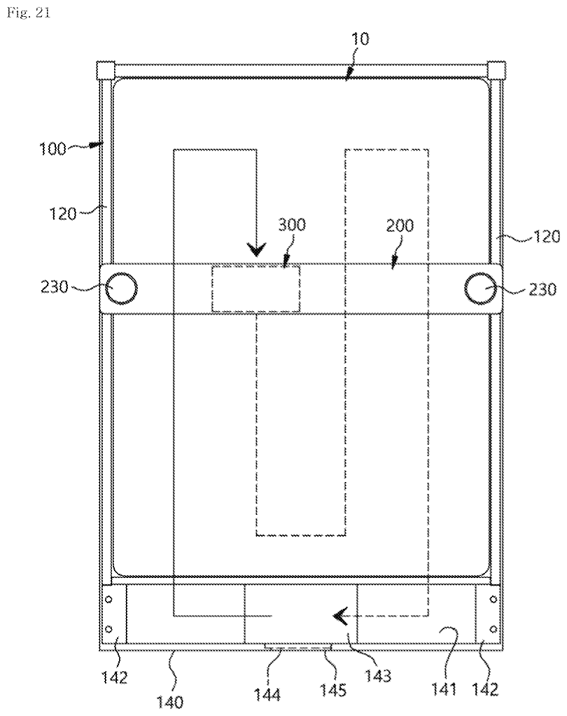

The cleaning module 300 is moved when the moving module 200 is fully moved forward or fully moved backward and the movement distance is determined in consideration of the suction ability of the cleaning module 300. That is, as shown in FIG. 21, while the moving module 200 and the cleaning module 300 are sequentially moved, the entire mattress 10 is uniformly cleaned.

Of course, cleaning module 300 and the moving module 200 may be simultaneously controlled to move left and right and move forward and backward, respectively. In this case, the speeds of cleaning module 300 and the moving module 200 are controlled in consideration of their routes, or the moving module 200 is controlled to repeatedly move forward and backward.

When the moving module 200 is moved, the operation speed of the wheel-driving motors 213 at the two side frames 210 or the rolling speeds of the rollable moving wheels 212 may be slightly different rather than completely the same.

If a speed difference is generated between the two side frames 210, as shown in FIG. 22, the top frame 220 is inclined at both sides with respect to the center portion when seen from above.

However, since the protrusions 214 are formed at the lower ends of the two side frames 210 and support the outer sides of the side walls 120 of the bed frame 100, the moving module 200 is not separated from the side walls 120 of the two side frames 210 and is not inclined such that poor operation is generated (i.e., in a configuration without rolling members 215).

Considering the rolling members 215 are disposed in the inners surface of the protrusions 214, even though the protrusions 214 come in contact with the side walls 120, the rolling members 215 come first in contact with the side walls 120 and roll, thereby removing any possible restriction. Accordingly, poor operation that the side frames 210 are restricted by the side walls 120 is prevented.

In particular, since the top frame 220 is installed to be rotatable with respect to the two side frames 210, even though the moving module 200 is inclined (i.e., twisted), the top frame 220 maximally reduces a speed difference while rotating with respect to the two side frames 210, whereby restriction and poor operation are prevented.

That is, when a speed difference is generated between the two side frames 210 and the moving module 200 is inclined to a side that is moved faster, the side frames 210 rotate by themselves while hitting against the outer surfaces of the side walls 120 of the bed frame 100, whereby poor operation due to getting caught (i.e., frictionally engaging the side walls 120) can be prevented.

This configuration is shown in FIGS. 22 and 23. That is, a side frame 210 is rotated and hits (i.e., contacts) the side frame 210 against the outer surface of a side wall 120 of the bed frame 100, and this rotation of the side frame 210 (i.e., via the rolling member(s) 215) prevents a restriction of movement of the side frame 210.

While the mattress 10 is cleaned, the UV-C emitter 330 of the cleaning module 300 may be operated in accordance with a setting or an operation by a user. Of course, the UV-C emitter 330 may be controlled to be independently operated without operation of suction unit 320.

When the UV-C emitter 330 is operated, UV-C is emitted to the surface of the mattress 10, whereby harmful insects or bacteria existing at the mattress 10 die out.

In particular, when the UV-C emitter 330 of the cleaning module 300 is operated, the moving module 200 is controlled to move slower than when only the suction unit 320 is operated.

That is, when the UV-C emitter 330 is operated, the moving module 200 is controlled to slowly move, whereby sterilization effect by UV-C can be sufficiently achieved.

When the mattress 10 finishes being cleaned by the operation control described above, the controller 400 controls the moving module 200 and the cleaning module 300 to return to the initial set positions.

As a result, the mattress care device of the present disclosure can prevent poor movement while the two side frames 210 are moved along the two side walls 20 of the bed frame 100, even if a speed difference is generated between the left and right sides when the moving module 200 is moved forward and backward.

That is, even though a speed difference is generated between the two side frames 210 and the moving module 200 is inclined to the faster side, the side frame 210 at the side rotates as much as hitting against the side wall 120 of the bed frame 100, whereby the side frame 210 can be prevented from being caught by the side wall 120 and corresponding poor movement can be prevented.

Further, according to the mattress care device of the present disclosure, since even though the side frames 210 hit against the side walls 120, they are not restricted (caught) by the protrusions 214 of the two side frames 210 and the rolling members 215 installed at the protrusions 214, so poor operation can be prevented.

Further, the mattress care device of the present disclosure can uniformly clean the entire mattress 10 even though the left-right width of the cleaning module 300 is smaller than the left-right width of the mattress 10.

That is, since the moving module 200 is installed to move forward and backward with respect to the mattress 10 and the cleaning module 300 is installed to move left and right with respect to the mattress 10, the size of the cleaning module 30 can be reduced.

In particular, it is possible to perform sufficient sterilization even by providing a minimum UV-C LED, whereby the manufacturing cost can be reduced.

The mattress care device of the present disclosure is not limited only to the structure of the above embodiment.

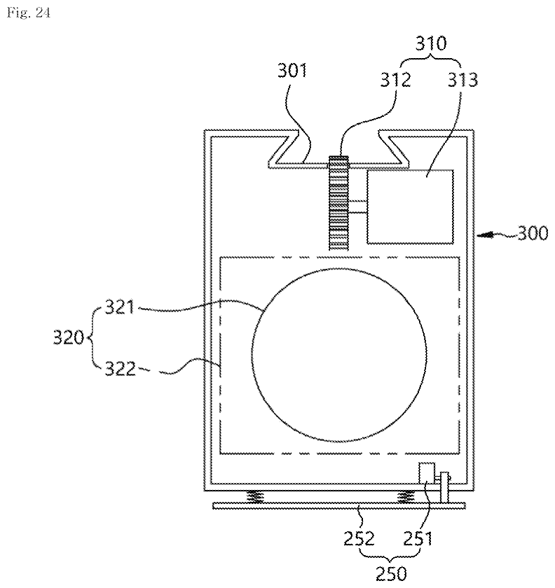

For example, the mattress care device of the present disclosure may include a vibrator 250 at the cleaning module 300 (see FIG. 24). That is, since the vibrator 250 is additionally provided, dirt, such as dust, on the surface of the mattress 10 can be separated to be easily suctioned while the surface of the mattress 10 over which the cleaning module 300 moves is hit.

The vibrator 250, as shown in FIG. 24, may include a vibration source 251, such as an ultrasonic oscillator or a vibration motor, and a vibration plate 252 that is vibrated up and down by operation of the vibration source 251.

The rollable moving wheels 212 installed on the bottoms of the two side frames 210 may be formed to surround the upper ends of the side walls 120 of the bed frame 100.

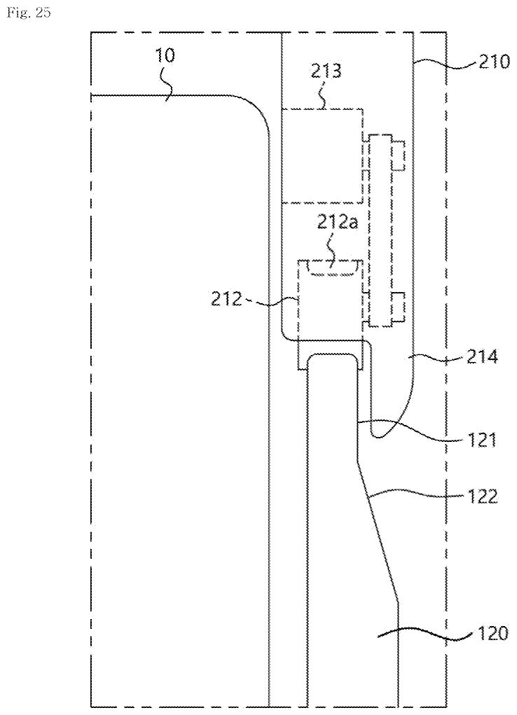

That is, as shown in FIG. 25, a recessed groove 212a in which the side wall 120 is accommodated is formed in the moving wheel 212. Accordingly, the two side frames 210 can accurately move along the side walls 120 even without adding the protruding ends 214 or the rolling members 215.

Of course, according to the structure in which the moving wheels 212 surround the upper ends of the side walls 120, the possibility of restriction, such as getting caught, is high when the moving module 200 is inclined (twisted) left and right due to a speed difference between both sides while moving.

However, since the top frame 220 of the moving module 200 can rotate with respect to the two side frames 210, such restriction can be prevented, so the moving wheels 212 can be formed to surround the upper ends of the side walls 120.

Accordingly, the mattress care device of the present disclosure can be achieved in various ways.

Various embodiments described herein may be implemented in a computer-readable medium using, for example, software, hardware, or some combination thereof. For example, the embodiments described herein may be implemented within one or more of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, other electronic units designed to perform the functions described herein, or a selective combination thereof. In some cases, such embodiments are implemented by the controller. That is, the controller is a hardware-embedded processor executing the appropriate algorithms (e.g., flowcharts) for performing the described functions and thus has sufficient structure. Also, the embodiments such as procedures and functions may be implemented together with separate software modules each of which performs at least one of functions and operations. The software codes can be implemented with a software application written in any suitable programming language. Also, the software codes can be stored in the memory and executed by the controller, thus making the controller a type of special purpose controller specifically configured to carry out the described functions and algorithms. Thus, the components shown in the drawings have sufficient structure to implement the appropriate algorithms for performing the described functions.

The present invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A mattress care device, comprising:
a bed frame including:
a first side wall;
a second side wall; and
an accommodation space formed between the first side wall and the second side wall, the accommodation space being configured to accommodate a mattress;
a moving module including:
a first side frame disposed on a top surface of the first side wall;
a second side frame disposed on a top surface of the second side wall; and
a top frame connected between the first side frame and the second side frame;
a cleaning module positioned on the moving module, the cleaning module being configured to:
move about the moving module in a horizontal direction; the horizontal direction being a width direction of the mattress; and
clean a top surface of the mattress; and
a controller configured to control an operation of the moving module and the cleaning module.

2. The mattress care device of claim 1, wherein a first side of the top frame is rotatably connected to the first side frame by a first hinge, and
wherein a second side of the top frame is rotatably connected to the second side frame by a second hinge.

3. The mattress care device of claim 2, wherein the first side of the top frame includes a first seat groove,
wherein the first side frame includes a first bending end engaging the first seat groove, and
wherein the first side of the top frame is coupled to the first bending end through the first hinge.

4. The mattress care device of claim 3, wherein the second side of the top frame includes a second seat groove,
wherein the second side frame includes a second bending end engaging the second seat groove,
wherein the second side of the top frame is coupled to the second bending end through the second hinge, and
wherein the first bending end faces the second bending end.

5. The mattress care device of claim 1, wherein the bed frame further includes:
a first side accommodation groove configured to accommodate the first side frame;
a second side accommodation groove configured to accommodate the second side frame; and
a storage groove configured to accommodate the top frame.

6. The mattress care device of claim 5, wherein the cleaning module includes a suction unit configured to provide a suction force against the mattress, the suction unit including:
an intake hole;
a fan motor disposed in the cleaning module and configured to provide the suction force; and
a dirt collection tub disposed in the cleaning module and fluidly connected to the fan motor, and
wherein the dirt collection tub is configured to collect foreign substances suctioned through the intake hole by the fan motor.

7. The mattress care device of claim 6, wherein the bed frame further includes:
a cleaner accommodation groove recessed in the storage groove, the cleaner accommodation groove being configured to accommodate the cleaning module; and
a door configured to open and close to provide a user access to the dirt collection tub.

8. The mattress care device of claim 1, wherein each of the first side frame and the second side frame includes:
a rollable moving wheel contacting the top surface of a respective one of the first side wall and the second side wall, the rollable wheel being configured to rotate with respect to the respective one of the first side wall and the second side wall; and
a wheel-driving motor configured to rotate the rollable moving wheel.

9. The mattress care device of claim 1, wherein each of the first side frame and the second side frame includes a protrusion protruding downward and facing an outer surface of a respective one of the first side wall and the second side wall, and
wherein each protrusion includes at least one rolling member, each rolling member being configured to rotate against an outer surface of the respective one of the first side wall and the second side wall.

10. The mattress care device of claim 1, wherein a bottom surface of the top frame includes a rack gear extending in a longitudinal direction of the top frame,
wherein the cleaning module includes a movement motor having a pinion gear, and
wherein the pinion gear is exposed from a top surface of the cleaning module and engages with the rack gear of the top frame to move the cleaning module with respect to the top frame.

11. The mattress care device of claim 10, wherein the top frame includes a guide rail formed on the bottom surface of the top frame in the longitudinal direction of the top frame,
wherein the rack gear is disposed within the guide rail, and
wherein the top surface of the cleaning module includes a guide groove, the guide rail being accommodated in the guide groove to engage the rack gear with the pinion gear.

12. The mattress care device of claim 1, wherein the cleaning module includes a UV-C emitter configured to emit ultraviolet-c (UV-C), and
wherein the UV-C emitter includes any one of a UV-C lamp and a UV-C LED.

13. The mattress care device of claim 12, wherein the controller is configured to control a first movement speed of the cleaning module when the UV-C emitter emits UV-C to be lower than a second movement speed when the UV-C emitter does not emit UV-C.

14. A mattress care device, comprising:
a bed frame including:
a first side wall extending along a longitudinal axis of the bed frame; and
a second side wall extending along the longitudinal axis of the bed frame;
a moving module extending along a horizontal axis of the bed frame, perpendicular to the longitudinal axis of the bed frame, the moving module including:
a first side frame;
a second side frame; and
a top frame connected between the first side frame and the second side frame; and
a cleaning module positioned on the moving module, the cleaning module being configured to:
move with respect to the moving module; and
clean a top of a mattress.

15. The mattress care device of claim 14, wherein a first side of the top frame is rotatably connected to the first side frame by a first hinge, and
wherein a second side of the top frame is rotatably connected to the second side frame by a second hinge.

16. The mattress care device of claim 14, wherein each of the first side frame and the second side frame includes:
a rollable moving wheel contacting a top surface of a respective one of the first side wall and the second side wall, the rollable wheel being configured to rotate with respect to the respective one of the first side wall and the second side wall; and
a wheel-driving motor configured to rotate the rollable moving wheel.

17. The mattress care device of claim 14, wherein each of the first side frame and the second side frame includes a protrusion protruding downward and facing an outer surface of a respective one of the first side wall and the second side wall, and
wherein each protrusion includes at least one rolling member, each rolling member being configured to rotate against an outer surface of the respective one of the first side wall and the second side wall.

18. The mattress care device of claim 14, wherein a bottom surface of the top frame includes a rack gear extending in a longitudinal direction of the top frame,
wherein the cleaning module includes a movement motor having a pinion gear, and
wherein the pinion gear is exposed from a top surface of the cleaning module and engages with the rack gear of the top frame to move the cleaning module with respect to the top frame.

19. The mattress care device of claim 18, wherein the top frame includes a guide rail formed on the bottom surface of the top frame in a longitudinal direction of the top frame,
wherein the rack gear is disposed within the guide rail, and
wherein the top surface of the cleaning module includes a guide groove, the guide rail being accommodated in the guide groove to engage the rack gear with the pinion gear.

20. The mattress care device of claim 14, wherein the cleaning module includes:
a suction unit configured to provide a suction force against the mattress, the suction unit including:
an intake hole;
a fan motor disposed in the cleaning module and configured to provide the suction force;
a dirt collection tub disposed in the cleaning module and fluidly connected to the fan motor, the dirt collection tub being configured to collect foreign substances suctioned through the intake hole; and
a UV-C emitter configured to emit ultraviolet-c (UV-C), and
wherein the UV-C emitter includes any one of a UV-C lamp and a UV-C LED.
